(19) United States
(12) Patent Application Publication
(10) Pub. No.: US 2002/0103174 A1
(43) Pub. Date: Aug. 1, 2002

(54) Use of eugenol, alone, and in combination with other chemopreventative agents as prophylaxis for cancers

(76) Inventors: Thomas J. Slaga, Golden, CO (US); Addanki P. Kumar, Denver, CO (US); William Alworth, New Orleans, LA (US)

Correspondence Address: DAVID G. HENRY, 900 Washington Avenue, P.O. Box 1470, Waco, TX, 76701, US

(21) Appl. No.: 10/004,105

(22) Filed: Dec. 4, 2001

Related U.S. Application Data
(63) Continuation-in-part of application No. 09/527,283, filed on Mar. 17, 2000, now abandoned.
(63) Continuation-in-part of application No. 09/777,151, filed on Feb. 5, 2001.
(63) Continuation-in-part of application No. 09/777,559, filed on Feb. 6, 2001, now abandoned.

(51) Int. Cl. A61K 31/56

(52) U.S. Cl. 514/169

(57) ABSTRACT

The use of eugenol, alone and in combination with 2-methoxyestradiol (2-ME) in the context of prostate cancer prophylaxes and treatment, and in the treatment and prevention of non-cancerous enlargement of prostate glands.

### CITATION TO PRIOR APPLICATION

This is a continuation-in-part with respect to U.S. application, Ser. No. 09/527283, filed MAR. 17, 2000 from which priority is claimed under 35 U.S.C. § 120 and under provisions of the Patent Cooperation Treaty. This is also a continuation-in-part with respect to U.S. applications Ser. Nos. 09/777,151 and 9/777,559 which also claim priority of U.S. Ser. No. 09/527283.

### BACKGROUND OF THE INVENTION

A. Field of the Invention

The present invention relates to the prevention and treatment of cancer through the use of chemopreventative agents.

B. Background of the Invention

Prostate cancer is the most common malignant transformation that occurs in men and its incidence is increasing at an alarming rate. Prostate cancer ranks as the most serious task facing both doctors and their male patients. Unfortunately, the major cause of death from prostate cancer comes in the hormone-refractory metastatic stage of the disease for which no treatment options are available at present.

Complicating the diagnosis and treatment of prostate cancer is the fact that prostate cancer remains latent and harmless in most individuals; clinically evident in many individuals, and virulent in still others. These variations in the expression of prostate concern make it even more difficult to derive efficacious treatment regimens. The therapies that are now available are associated with side effects which include impotence in about 59.9% men after 18-months of prostatectomy.

Over the past several years chemoprevention has been established as a meaningful approach to control malignancy. Chemopreventative approaches use either natural and/or synthetic compounds to intervene in the early pre-cancerous stages of carcinogenesis before cancer actually begins and thus having a greater chance of total cure. Like most cancers, prostate cancer is a complex process involving alterations in the balance between cell proliferation and cell death and accumulation of a series of genetic changes. Such an alteration exists in prostate cancer and in benign prostate hyperplasia (BPH). In BPH, the apoptotic index (percentage of cells undergoing apoptosis) is significantly reduced in the epithelial cells, basal cells and the stromal cells, while at the same time the proliferative index is significantly increased in all three cell types. These changes occur over a long period of time thus providing a large window of opportunity to i) prevent its induction; ii) inhibit the development of pre-invasive or invasive neoplasia and iii) its progression by using chemopreventative agents.

It is well known that prostate cancer is highly heterogeneous in that the tumor contains a population of both androgen-dependent and independent cells and also cells at different stages of transition between androgen-dependence and independence. Androgen ablation is, to date, the therapy of choice and is widely used to inhibit prostate cancer in the initial stages of prostate concern; however, the likelihood of recurrence of tumors (androgen-independent) limits this therapeutic approach. The average survival time after failing androgen-ablation therapy is about 12 months.

Prostate specific antigen (PSA) has traditionally been used as a tumor marker to detect prostate cancer. Many studies have demonstrated that most of the patients with established benign prostatic hyperplasia (BPH) undergoing prostatectomy have abnormal levels of PSA. 10 years after surgery approximately 25% of patients with PSA levels between 4-10 ng/ml; 70% between 10-20 ng/ml and 72% for men with PSA levels greater than 20 ng/ml develop prostate cancer. In addition, men with PSA levels of 2-4 have a 30% chance of developing prostate cancer after 5 years.

Also relevant to the inventions disclosed herein is the fact that enlarged prostate glands tend to become cancerous, though they are not necessarily cancerous at the time that their enlargement becomes detectable.

### SUMMARY OF THE INVENTION

In view of the above, there is a dire need for more effective preventative and therapeutic approaches in dealing with prostate cancer specifically and, of course, all cancers for which there is no presently available and reliable cure. It is also of great importance to provide a means and method by which enlarged, non-cancerous prostate glands may be treated (and thereby reduced in size) as an additional approach to preventing prostate cancer, as well as to address the other side effects of enlarged prostate glands which are independent of the dangers of cancer (urinary problems and erectile dysfunction being worthy of note in this regard).

It is, therefore, an object of the present invention to provide a new modality for the prevention of cancer.

It is another object of the present invention to provide a new modality for the treatment of cancer.

It is another object of the present invention to provide a new modality for the treatment of prostate cancer.

It is another object of the present invention to provide a method by which the known substance of eugenol may be employed in a new and unobvious manner in the prevention and/or treatment of cancers, including prostate cancer.

It is another object of the present invention to provide a method by which the known substance of eugenol may, in combination with synergistic compounds, including 2-ME, be employed in the prevention and/or treatment of cancers, including prostate cancer.

It is another object of the present invention to provide a new modality for the treatment of non-cancerous enlarged prostate glands.

It is another object of the present invention to provide a new modality for the prevention of enlargement of prostate glands.

It is another object of the present invention to provide a method by which the known substance of eugenol may be employed in a new and unobvious manner in the prevention and/or treatment of non-cancerous enlarged prostate glands.

It is another object of the present invention to provide a method by which the known substance of eugenol may, in combination with synergistic compounds, including 2-ME, be employed in the prevention and/or treatment of enlarged, non-cancerous prostate glands.

In satisfaction of these and related objects, disclosed and claimed herein is the use of eugenol, alone and in combination with 2-methoxyestradiol (2-ME) in the context of prostate cancer and enlarged prostate prophylaxes and treatment.

Eugenol is a major component of the essential oils from bay leaves and the buds of cloves (Eugenia Caryophyllata). It is widely used as a flavoring agent in food products, pharmaceuticals products and also as an analgesic in dentistry. However, nothing has been heretofore known about eugenol's capacity for preventing and treating cancer..

The use of eugenol either alone or in combination with 2-ME offers the following important advantages: i) since eugenol has been used as an analgesic successfully in the dentistry, toxicity is unlikely; (ii) cell cycle analysis data indicated that eugenol inhibited the growth of suspect cells without any significant alterations in the cells cycle profile, thereby indicating a different mechanism of action than that of 2-ME when used in the same context; (iii) yet, eugenol demonstrated synergistic activity with 2-ME. The present inventors have shown that 2-ME inhibits the growth of cells by inducing apoptosis and blocking cells in G2/M phase. Therefore induction of cell death pathway by 2-ME and growth inhibition by eugenol through a different pathway presents a tremendous new weapon for use in preventing and combating cancer.

### DETAILED DESCRIPTION OF THE PREFERRED EMBODIMENT

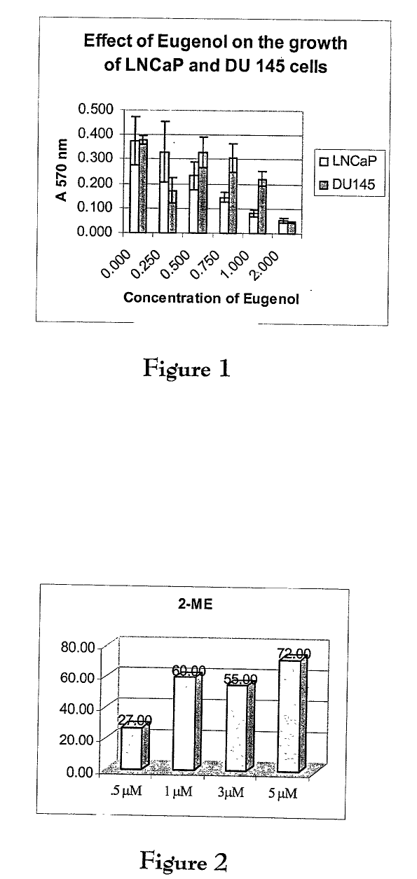

The present inventors have used androgen-dependent (LNCaP) and androgen-independent (DU145) human prostate cancer cell lines to investigate the effect of eugenol and isoeugenol. These cells were treated with different concentrations of this compound (0.5, 1, 3, 5 and 10 μM). Cell growth was monitored every 24 h by counting the increase in the cell number using trypan blue exclusion assay. These results were also confirmed by using cell proliferation assay kit. As shown in FIG. 1, eugenol inhibited the growth of LNCaP cells significantly. A concentration of approximately 0.75 μM was necessary to see 50% inhibition of growth of LNCaP cells whereas a concentration of more than 2 μM was necessary to see similar effect in DU145 cells.

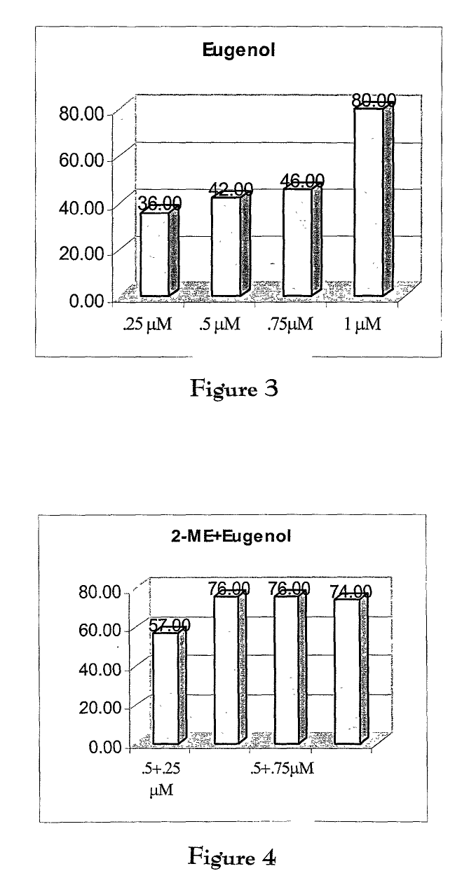

Referring to FIGS. 2, 3 and 4, also tested was the effectiveness of eugenol in combination with 2-ME. Cells were treated with either eugenol alone (FIG. 2-0.25, 0.5, 0.75 or 1 μM) or 2-ME alone (FIG. 3-0.5, 1, 2 or 3 μM) or both (FIG. 4-0.25, 0.5, 0.75 or 1 μM of eugenol along with 0.5 μM of 2-ME). Cell growth was measured following 72 h of treatment as described above. As shown in the figures, 0.5 μM of 2-ME inhibited the growth of LNCaP cells by about 27%; 0.25 μM of eugenol inhibited the growth by about 36%. However, as shown in FIG. 4, combining both the agents showed more than 70% inhibition indicating additive activity.

Based on the above cell culture data, a pilot study was conducted to evaluate the efficacy of 2-ME and Eugenol singly (monotherapy) and in combination (combination therapy) using LNCaP human prostate tumor xenografts. Male nude athymic mice (Harlan) were injected sub-cutaneously in the flank region with 1×10⁷ LNCaP prostatic adenocarcinoma cells suspended in 0.2 ml saline. Mice were grouped into nine groups of ten animals each, and treatment was initiated in all groups. The compounds were delivered in 0.2 ml of dosing solution per 20 g of body weight and all doses were body-weight adjusted. Stock solution of 2-ME was prepared in N,N-dimethylacetaminde (DMA) at a concentration of 250 and 83.3 mg/ml. These stock solutions were diluted with hydroxypropyl-β-cyclodextrin (HBC) on each day of treatment to provide appropriate dosing solutions in 40% HBC containing 3% DMA. Eugenol was mixed with corn oil on each day of dosing. Both the compounds were protected from light and stored at 4° C.

Group I Vehicle control (control mice received i.v injections of 3% DMA in 40% HBC twice per week).

Group II 25 mg/kg 2-ME twice a week.

Group III 75 mg/kg 2-ME twice a week.

Group IV 500 mg/kg eugenol twice a week.

Group V 1000 mg/kg eugenol twice a week.

Group VI 25 mg/kg 2-ME and 500 mg/kg eugnol.

Group VII Daily oral doses of 3% DMA/40% HBC vehicle.

Group VIII Daily oral dose of 2-ME at 25 mg/kg.

Group IX Daily oral dose of 2-ME at 75 mg/kg.

Tumors were measured twice a week beginning on day 12. The estimated tumor weight (mg) was calculated using the formula w²×½; where w is the width and 1 length in mm of tumor. Each animal was euthanized when its carcinoma reached 1 g or more and was considered cancer death.

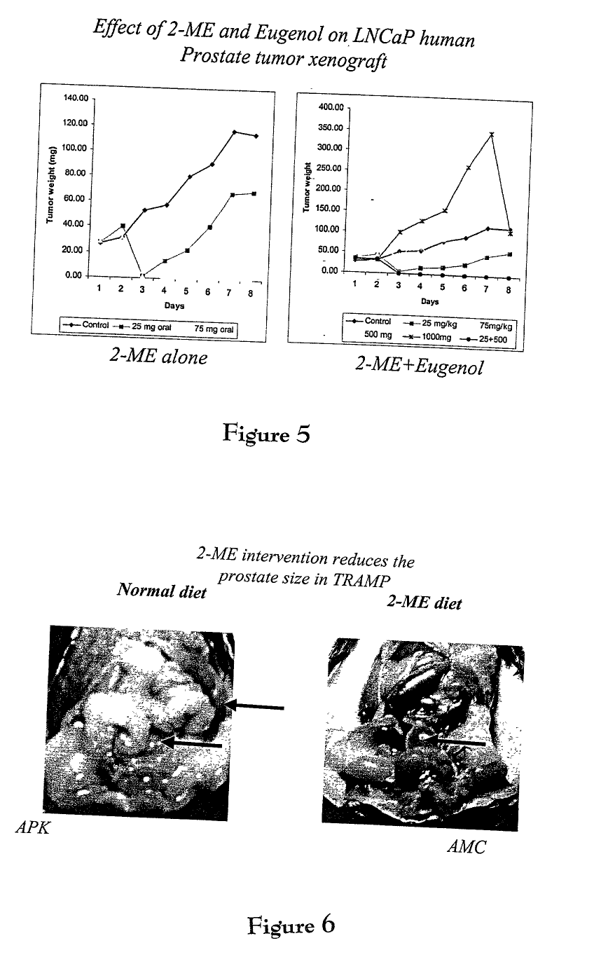

2-ME and eugenol were administered to male athymic nude mice immediately after LNCaP cells were injected so as to allow the greatest opportunity for blocking the emergence of tumors. Observations were of a higher percentage of CR (complete regression of tumors) responses for mice that received oral 2-ME. There were seven and six such responses to the 25 and 75 mg/kg oral regimens respectively (groups 9 and 10). As shown in FIG. 5, the tumor weight was also reduced in the groups of mice receiving 2-ME.

Eugenol by it self did not affect the tumor weight under the conditions tested (500 and 1000 mg/kg body weight), but the group of animals that received (500 mg eugenol and 25 mg of 2-ME) showed reduction in the tumor weight. Out of six survivors in the combination group, five showed complete regression of tumors and one showed stable/progressive response.

The i.v treatment with 2-ME and oral treatments with the 3% DMA/40% HBC vehicle produced almost negligible body-weight losses ranging from 1.2-2.8%. Daily oral administration of 2-ME caused approximately 9% maximum group mean weight loss on day 13 at both the 25 and 75 mg/kg dosing levels. This weight loss is well within the acceptable range, since NCI defines the maximum tolerated dose (MTD) as one that causes less than 20% group mean body-weight loss. The combination regimen produces an acceptable maximum group mean weight loss of 11.1% on day 2.

Also investigated was the efficacy of 2-ME in preventing the development of prostate cancer using transgenic adenocarcinoma of mouse prostate (TRAMP) model. These mice develop metastatic adenocarcinomas between 10 and 20 weeks of age that are partially androgen-independent (65%). In addition, these adenocarcinomas develop in the dorsolateral lobe of the mouse prostate that is considered most analogous to the peripheral zone where human prostate cancer originates. Hence, the results obtained from this model may directly be translated to the human situation. This model system exhibits progressive forms of prostatic disease that histologically resembles human prostate cancer, ranging from mild intraepithelial hyperplsia to large mutlinodular malignant neoplasia. Greenberg and colleagues have shown that the TRAMP mice develop hyperplasia between 8-12 weeks of age; neoplasia between 15-24 weeks and metastasis between 24-36 weeks of age.

Initially, the mice were fed at the age of 8 weeks to see the effect on prostatic hyperplasia. These mice were sacrificed when they reached 24 weeks of age.

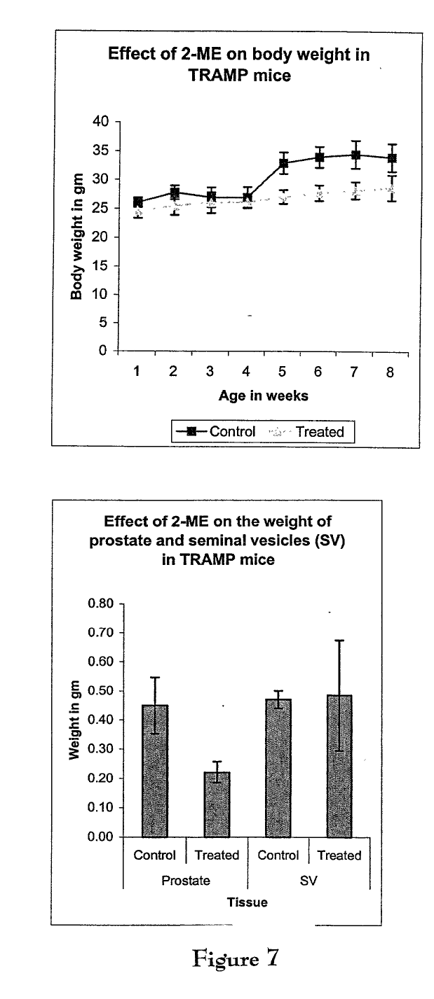

Preliminary results indicate drastic reduction in the size of prostate gland and seminal vesicles in the group of mice that were on 2-ME diet when compared to the mice on normal diet (see FIGS. 6 and 7).

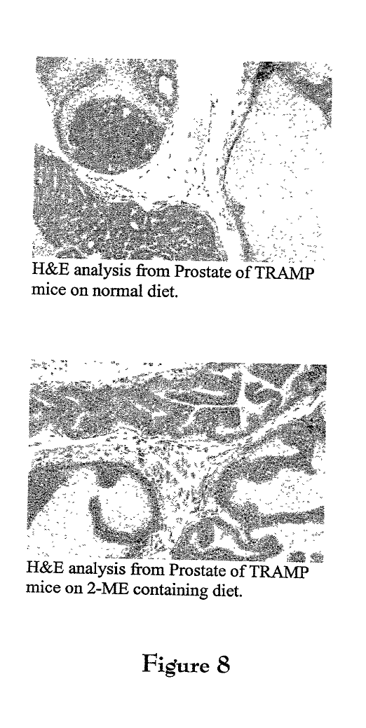

Referring to FIG. 8, histological analysis of the prostate from 24 week Tramp mouse displays major epithelial proliferation in a characteristic cribriform pattern; hyperchromatic nuclei; mitotic figures and apoptotic bodies. As mentioned, following feeding on diet coposed of 2-ME for 16 weeks, starting at 8th week, the volume of the prostate gland is less compared to the control and histological analysis indicates that the glands are composed of a columnar epithelium with round to oval nuclei with no indications of neoplasia.

Of great significance is the fact that these results indicate that 2-ME can be used for the treatment of enlarged, non-cancerous prostate gland which may reduce the incidence of prostate cancer. Though preliminary, these studies have an immense potential for developing 2-ME in prevention, intervention and/or in regression during prostate carcinogenesis. Prevention of enlarged prostate glands through the use of the indicated agents also naturally flows from the reduction of existing enlargement.

Although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

We claim:

1. A method for inhibiting the growth of cancerous and precancerous cell populations comprising the step of applying a therapeutic amount of eugenol to said cell population for a sufficient time to observe arrest of population growth.

2. The method of claim 1 further comprising the step of applying a therapeutic amount of 2-ME in combination with said amount of eugenol to said cell population.

3. The method of claim 1 wherein said cancerous cell populations are prostate cancer cells.

4. A therapeutic agent useful in the prevention and treatment of cancerous tumors comprising eugenol and 2-methoxyestradiol.

5. The therapeutic agent of claim 4 wherein said cancerous tumors are prostate cancer tumors.

6. A therapeutic agent for the prevention and treatment of enlarged, non-cancerous prostage tissues comprising a therapeutic dossage of 2-methoxyestradiol.

7. The therapeutic agent of claim 6 further comprising a therapeutic dossage of eugenol.

8. A method for preventing the development of, or for treating existing enlarged, non-cancerous prostate glands in mammals comprising the steps of administering a therapeutic dossage of 2-methoxyestradiol.

9. The method of claim 8 further comprising the administration of a therapeutic dossage of eugenol in combination with said 2-methoxyestradiol.
